(19) United States
(12) Patent Application Publication
(10) Pub. No.: US 2006/0039933 A1
(43) Pub. Date: Feb. 23, 2006

(54) Method for treatment of insect bites

(76) Inventors: John O. Cram, Hilton Head Island, SC (US); Miguel Castillo, Hilton Head Island, SC (US)

Correspondence Address: WILLIAM H. EILBERG, THREE BALA PLAZA, SUITE 501 WEST, BALA CYNWYD, PA, 19004, US

(21) Appl. No.: 11/203,697

(22) Filed: Aug. 15, 2005

Related U.S. Application Data
(60) Provisional application No. 60/602,807, filed on Aug. 19, 2004.

(51) Int. Cl. A61K 9/00; A61K 31/22; A61K 31/01

(52) U.S. Cl. 424/400; 514/546; 514/762

(57) ABSTRACT

A method of treating wounds caused by the bites of small insects or jellyfish includes applying a water-based wax emulsion to the wound. When the emulsion dries, it forms a thin, protective film over the wound. Application of the emulsion has been found to provide temporary and immediate relief of pain and/or itching. The protective film shields the wound, and therefore aids in healing. The emulsion can be made of any of a variety of waxes, and the emulsion can be formed with long-chain alcohol or fatty acid soaps, such as oleic acid or triethanolamine, or any other materials that form a water-wax emulsion.

### CROSS-REFERENCE TO PRIOR APPLICATION

Priority is claimed from U.S. Provisional Patent Application Ser. No. 60/602,807, filed Aug. 19, 2004.

### BACKGROUND OF THE INVENTION

The present invention is a method of treating minor wounds of the skin caused by the bites of small insects or jellyfish, and provides temporary relief of pain and itching, while promoting healing.

It has been known to treat skin wounds caused by the bites of small insects or jellyfish with products that are similar to a clear nail polish. Such products generally contain volatile and flammable solvents, as well as other materials that serve to form a protective coating. An example of such a product is the material sold under the trademark Chigarid, which is available from Colgin Companies, of Dallas, Tex.

The present invention provides a method for treatment of wounds caused by bites of small insects, such as chiggers or mites, as well as the bites of jellyfish. The invention avoids the use of volatile or flammable solvents. The non-flammable material used in the present invention has been found to work well in relieving pain and itching, and in promoting healing.

### SUMMARY OF THE INVENTION

The present invention comprises treating a minor wound of the skin, caused by the bite of a small insect or jellyfish, with a water-based wax emulsion.

The water-based wax emulsion is applied over the wound. When the emulsion dries, it forms a thin protective film over the wound. Application of the emulsion has been found to provide temporary and immediate relief of pain and/or itching. The protective film shields the wound, and therefore aids in healing.

The water-based wax emulsion used in the present invention can be made of any of a variety of types of wax, such as carnauba wax, paraffin, microcrystalline wax, bees wax, montan wax, candelilla wax, shellac, or other waxes, or any combination of the foregoing. The material used to form the emulsion may be long-chain alcohol or fatty acid soaps, and may include substances such as oleic acid or triethanolamine, or any other materials that form a stable water/wax emulsion.

The water-based wax emulsion used in the present invention does not contain any organic solvents or flammable liquids. The use of the emulsion therefore constitutes an improvement over the prior art, in which the use of such flammable materials is common. Among other things, the use of a non-flammable wax emulsion makes it practical to store the material conveniently in virtually any container, including a plastic container.

The present invention therefore has the primary object of providing a method of treating minor wounds of the skin caused by bites of small insects or jellyfish.

The invention has the further object of providing a method of treating insect and jellyfish bites, without requiring the use of volatile organic solvents, or other flammable materials.

The invention has the further object of providing a material for treatment of insect and jellyfish bites, which material can be safely and conveniently stored in plastic containers.

The invention has the further object of providing a method of treating wounds caused by insect and jellyfish bites, wherein the method provides temporary but immediate relief of pain and/or itching, and wherein the method also promotes healing of the wound.

The invention has the further object of providing a method of treating wounds caused by insect and jellyfish bites, wherein the method uses a material that is not likely to be abused by those seeking to become intoxicated.

The reader skilled in the art will recognize other objects and advantages of the present invention, from a reading of the following detailed description of the invention, and the appended claims.

### DETAILED DESCRIPTION OF THE INVENTION

The present invention comprises the treatment of wounds, caused by the bites of small insects or jellyfish, with a water-based wax emulsion. It has been found that the wax emulsion can be used as a skin medication to treat such wounds. The emulsion dries to form a substantially waterproof, thin, protective film over the wound.

The water-based wax emulsion may be applied to wounds caused by the bites of any of a variety of small insects, such as chiggers or red bugs, mites, sand flies, fleas, or other small insects. It is also useful on wounds caused by the bites of jellyfish.

The wax emulsion, when applied, has been found to provide immediate and temporary relief of pain and itching. The product forms a protective film over the wound, and therefore promotes healing.

The wax emulsion can be made from many possible types of wax. These types include carnauba wax, paraffin, microcrystalline wax, bees wax, montan wax, candelilla wax, shellac, or similar types of wax, or any combination of the foregoing.

The above-described waxes, which are insoluble in water, are emulsified with long-chain alcohol or fatty acid soaps, which reduce the surface tension at the interface of the suspended wax particles because of the solubility properties of their molecules.

The materials used to form the emulsion could be, for example, oleic acid or triethanolamine, or any other materials that form a water/wax emulsion. This allows the waxes to remain in a stable solution or emulsion. The invention is not limited to the specific examples given above.

When the material is applied to the wound, and when the water in the emulsion evaporates, the wax or waxes are left in a coherent water-resistant smooth film which covers the wound. This film prevents or stops itching, protects the wound, and therefore aids in healing.

The present invention completely eliminates the problems associated with volatile and flammable solvents, by using inert waxes and a soap-based emulsification system. Among other things, due to its inertness, the material will not readily react with other materials, including container materials, and therefore can conveniently be stored in plastic containers or bottles.

While wax emulsions have been known in the prior art, it has not been known to use such materials for treating wounds caused by insect bites or the like.

The wax emulsion used in the present invention may be packaged in a small bottle, containing one or two fluid ounces, with an applicator similar to what is used with a bottle of mercurochrome or iodine. The material can be packed in almost any type of plastic bottle. Unlike the case of some organic solvents, it is not necessary to store the wax emulsion in a glass container.

The present invention also has an important advantage with regard to public health. As noted above, the products used in the prior art include volatile organic solvents, which are often abused by young persons and others who are seeking to become intoxicated. The water-wax emulsion used in the present invention, by contrast, cannot be abused in this way.

The invention can be modified in various ways. The wax used in making the emulsion is not limited to the materials listed above, but can include other materials not specifically mentioned. The substances used to form the emulsion can also be varied. These and other modifications, which will be apparent to the reader skilled in the art, should be considered within the spirit and scope of the following claims.

What is claimed is:

1. A method of treating a wound caused by a bite of a small insect or jellyfish, comprising applying, to a site of a wound, a water-based wax emulsion.

2. The method of claim 1, further comprising the step of allowing the emulsion to dry, so as to form a substantially waterproof, thin film.

3. The method of claim 1, wherein the emulsion is selected to contain a wax which is selected from the group consisting of carnauba wax, paraffin, microcrystalline wax, bees wax, montan wax, candelilla wax, shellac, or any combination of the foregoing.

4. The method of claim 1, wherein the emulsion is formed with a long-chain alcohol or a fatty acid soap.

5. The method of claim 3, wherein the emulsion is formed with a long-chain alcohol or a fatty acid soap.

6. The method of claim 1, wherein the emulsion is formed with a material selected from the group consisting of oleic acid and triethanolamine.

7. The method of claim 1, wherein the water-based wax emulsion is selected to be free of flammable solvents.

8. The method of claim 1, further comprising the step of storing the water-based wax emulsion in a plastic container.

9. A method of treating a wound caused by a bite of a small insect or jellyfish, comprising applying, to a site of a wound, a water-based wax emulsion, wherein the emulsion is selected to contain a wax which is selected from the group consisting of carnauba wax, paraffin, microcrystalline wax, bees wax, montan wax, candelilla wax, shellac, or any combination of the foregoing, and allowing the emulsion to dry, so as to form a substantially waterproof, thin film, wherein the emulsion is selected to be free of flammable solvents.

10. The method of claim 9, wherein the emulsion is formed with a material selected from the group consisting of long-chain alcohols and fatty acid soaps.

11. The method of claim 9, wherein the emulsion is formed with a material selected from the group consisting of oleic acid and triethanolamine.

12. The method of claim 9, further comprising the step of storing the water-based wax emulsion in a plastic container.

13. A method of treating a wound caused by a bite of a small insect or jellyfish, comprising forming a water-based wax emulsion, the emulsion containing wax selected from the group consisting of carnauba wax, paraffin, microcrystalline wax, bees wax, montan wax, candelilla wax, shellac, or similar types of wax, or any combination of the foregoing, applying the emulsion to a wound, and allowing the emulsion to dry so as to provide a protective film over the wound.

14. The method of claim 13, wherein the emulsion is selected to include a material selected from the group consisting of long-chain alcohols and fatty acid soaps.

15. The method of claim 13, wherein the emulsion is selected to contain a material selected from the group consisting of oleic acid and triethanolamine.

16. The method of claim 13, further comprising the step of storing the water-based wax emulsion in a plastic container.
